(19) United States
(12) Patent Application Publication
(10) Pub. No.: US 2024/0216210 A1
(43) Pub. Date: Jul. 4, 2024

(54) SKIN CARE DEVICES

(71) Applicant: AGE SCIENCES, INC. dba PMD BEAUTY, Draper, UT (US)

(72) Inventor: Sam ALEXANDER, Alpine, UT (US)

(21) Appl. No.: 18/408,024

(22) Filed: Jan. 9, 2024

Related U.S. Application Data
(63) Continuation of application No. 16/270,479, filed on Feb. 7, 2019.

(51) Int. Cl. A61H 7/00; A46B 13/02; A46B 15/00; A47K 7/02; A61H 23/00; A61L 31/02; A61L 31/14; A61N 1/40; A61N 5/06

(52) U.S. Cl. CPC A61H 7/005; A46B 13/023; A46B 15/0075; A47K 7/028; A61H 23/00; A61L 31/028; A61L 31/14; A61N 1/40; A61N 5/0616; A61H 2201/0165; A61H 2201/0207; A61H 2201/0214; A61H 2201/10; A61H 2201/169; A61N 2005/0659; A61N 2005/0661; A61N 2005/0663

(57) ABSTRACT

The present disclosure is drawn to skin care devices. An example skin care device can include skin-rejuvenation head with a first external region including an array of skin-cleansing bristles and a second external region including an auxiliary skin-treatment appliance of a different type than the skin-cleansing bristles, e.g., an LED phototherapy appliance. The skin-rejuvenation head can include a vibrational motor positioned therein to provide vibratory oscillation to the skin-rejuvenation head.

The present application is a continuation of U.S. patent application Ser. No. 16/270,479, filed on Feb. 7, 2019, which is incorporated herein by reference.

### BACKGROUND

Skin care is a rapidly evolving industry where treatment devices, skin care formulations, and related treatment protocols are being developed on nearly a continuous basis. Dermal treatment to promote or prolong the appearance of healthy skin, particularly facial skin, can complicated, as there are many choices available to consumers and some approaches tend to work better than others for a given skin care goal. For example, skin cleansing and treatment for antimicrobial purposes, e.g., acne, may demand a different group of treatment protocols than skin tightening. With effective treatments sometimes finding improvement with multifaceted approaches, providing skin care devices, systems, and methods that are effective and convenient for achieving a given skin care goals can beneficial.

### BRIEF DESCRIPTION OF THE DRAWINGS

FIG. 1 illustrates multiple views of an example skin care device in accordance with the present disclosure;

FIG. 2 illustrates an exploded view of an example skin care device in accordance with the present disclosure;

FIG. 3 illustrates a cross-sectional view of example skin care device in accordance with the present disclosure;

FIGS. 4A and 4B illustrates multiple views of a skin-rejuvenation head and a neck portion of a handle of an example skin care device in accordance with the present disclosure;

FIGS. 5A and 5B illustrates multiple views of an alternative skin-rejuvenation head and a neck portion of a handle of an example skin care device in accordance with the present disclosure;

FIGS. 6A and 6B illustrates multiple views of an alternative skin-rejuvenation head and a neck portion of a handle of an example skin care device in accordance with the present disclosure;

FIGS. 7A and 7B illustrates multiple views of an alternative skin-rejuvenation head and a neck portion of a handle of an example skin care device in accordance with the present disclosure; and

FIGS. 8A and 8B illustrates multiple views of alternative skin-rejuvenation head and a neck portion of a handle of an example skin care device in accordance with the present disclosure.

### DETAILED DESCRIPTION

The present disclosure is drawn to skin care devices, as well as related systems and methods. In one example, a skin care device can include a skin-rejuvenation head including a first external region and a second external region thereon. The first external region can include an array of skin-cleansing bristles and the second external region can include an auxiliary skin-treatment appliance of a different material and type than the skin-cleansing bristles. The skin-rejuvenation head can also include a vibrational motor providing vibratory oscillation to both the skin-cleansing bristles and the auxiliary skin-treatment appliance. The skin care device can also include a support rod mechanically coupled to the skin-rejuvenation head. The skin care device can also include a handle with a neck portion and a grip portion. The support rod can be coupled to the handle, with the neck portion being proximal to the skin-rejuvenation head and the grip portion being distal to the skin-rejuvenation head. The neck portion can include an energy dampening sleeve that surrounds the support rod, leaving a gap around the support rod. In one example, the energy dampening sleeve can surround the support rod leaving an air gap around the support rod within the neck portion, and the support rod can be connected to the handle within the grip portion. Thus, the mechanical connection support rod between the skin-rejuvenation head and the grip portion of the handle can pass through the neck portion without contact or at least significant contact with the energy dampening sleeve. The energy dampening sleeve, thus, can be formed so that it does not provide enough structural support to support the skin-rejuvenation head in an upright position relative to the handle without the presence of the support rod. That is not to say that the energy dampening sleeve provides no structural support, but rather, its purpose is to provide energy dampening, such as with a soft rubber material, e.g., silicone rubber, and thus, may not be present entirely purposes of structural support. The combination of the support rod and the energy dampening sleeve, leaving a gap therebetween along at least part of the neck portion, can work together to provide good support between the handle and the skin-rejuvenation head, while provide acceptable energy dampening to the user holding the grip portion during use. This can be the case even in instances of high intensity or high frequency vibrational oscillation from the vibrational motor. In further detail, the skin care device can include a rubber sleeve that covers at least part of the grip portion, the neck portion, and at least part of the skin-rejuvenation head. In this configuration, the energy dampening sleeve at the neck portion can be part of the rubber sleeve, e.g., a continuous sleeve. However, in this example, the energy dampening sleeve can have an average outer wall thickness that is greater than the average outer wall thickness of rubber sleeve at the skin-rejuvenation head, the grip portion, or both. In further detail with regard to energy dampening between the vibrational motor in the skin-rejuvenation head and the grip portion of the handle, the neck portion can include a narrow a cross-sectional area compared to a cross-sectional area of the grip portion. For example. The cross-sectional area at least at one location along the neck portion can be from 4 to 15 times smaller in area than at least one a cross-sectional area of the grip portion.

In another example, a skin care device can include skin-cleansing bristles at a first external region, a semi-precious stone skin-treatment pad at a second external region, and a vibrational motor positioned to provide vibratory oscillation to both the skin-cleansing bristles and to the semi-precious skin-treatment stone pad. In one example, the semi-precious skin-treatment stone pad can be temperature controllable as a heating pad, a cooling pad, or both. In another example, the semi-precious skin-treatment stone pad can include quartz, e.g., rose quartz or another type of quartz that may be rich in metal and/or mineral content. Thus, for example, a quartz stone pad, such as rose quartz, can be associated with a heat conductor to provide heat to a skin surface through the rose quartz stone pad. In another example, the semi-precious skin-treatment stone pad can include jade of any type, e.g., nephrite, jadeite, and/or xiuyan. In one example, a jade stone pad can be associated with a cooling source to provide cooling to a skin surface through the jade stone pad. The skin-cleansing bristles, the semi-precious skin-treatment stone pad, and the vibrational motor can be integrated as part of a skin-rejuvenation head that is attached to a separate handle. In one example, the handle can be configured to dampen vibrational energy introduced at the skin-rejuvenation head, as described previously.

In another example, a skin care device can include skin-cleansing bristles at a first external region, a metal skin-treatment pad at a second external region, and a vibrational motor positioned to provide vibratory oscillation to both the skin-cleansing bristles and to the metal skin-treatment pad. The metal skin-treatment pad can include a metal selected from silver, gold, copper, or an alloy thereof. The metal skin-treatment pad can also be temperature controllable as a heating pad, a cooling pad, or both, e.g., heat on/heat off, variable heat, cooling on/cooling off, variable cooling, heating on/cooling on/heat and cooling off, variable heating and/or cooling, etc. The metal skin-treatment pad includes a substrate, e.g., plastic, another metal or alloy, etc., that can be silver-plated, copper-plated, gold-plated, or plated with an alloy thereof. The metal skin-treatment pad can be associated with a heat conductor to provide heat to a skin surface through the metal skin-treatment pad. In further detail, the metal skin-treatment pad can be associated with a cooling source to provide cooling to a skin surface through the metal skin-treatment pad. In another example, the skin-cleansing bristles, the metal heating pad, and the vibrational motor are all integrated as part of a skin-rejuvenation head, and wherein the head is attached to a handle. In one example, the handle can be configured to dampen vibrational energy introduced at the skin-rejuvenation head, as described previously.

In another example, a skin care device can include skin-cleansing bristles at a first external region, an exfoliation pad at a second external region, and a vibrational motor positioned to provide vibratory oscillation to both the skin-cleansing bristles and to the exfoliation pad. The exfoliation pad can include a substrate with aluminum oxide particulates embedded in the substrate. The aluminum oxide particulates can include aluminum oxide crystals having an average particle size to provide an 80 grit to 300 grit exfoliation surface. The exfoliation surface can alternatively provide an exfoliation surface from 150 grit to 250 grit, or from 80 grit to 150 grit, for example. The substrate can, for example, be a plastic substrate, and the aluminum oxide particulates can be embedded therein by thermally softening the substrate and pressing the aluminum oxide particulates into the substrate, e.g., to hold the aluminum oxide particulates in place. The skin-cleansing bristles, the exfoliation pad, and the vibrational motor are all integrated as part of a skin-rejuvenation head, and wherein the head is attached to a handle. In one example, the handle can be configured to dampen vibrational energy introduced at the skin-rejuvenation head, as described previously.

In another example, a skin care device can include skin-cleansing bristles at a first external region, a soft porous scrubber at a second external region, and a vibrational motor positioned to provide vibratory oscillation to both the skin-cleansing bristles and to the soft porous scrubber. The soft porous scrubber can include an anti-microbial metal associated therewith. The anti-microbial metal can include elemental silver, elemental copper, elemental gold, alloys of silver, alloys of copper, alloys of gold, ionic silver, ionic copper, ionic gold, a silver compound, a copper compound, a gold compound, or combinations thereof. The anti-microbial metal can be adsorbed on a surface of the soft porous scrubber, and/or impregnated within the soft porous scrubber. In another example, the anti-microbial metal can be in the form of a silver salt that is applied as a silver salt solution and then dried. The soft porous scrubber can be, for example, a natural loofah or a natural sponge. In another example, the soft porous scrubber can be a synthetic loofah, a synthetic sponge, a synthetic pouf, or a synthetic mesh. The skin-cleansing bristles, the soft porous scrubber, and the vibrational motor are all integrated as part of a skin-rejuvenation head, and wherein the head is attached to a handle. In one example, the handle can be configured to dampen vibrational energy introduced at the skin-rejuvenation head, as described previously.

In another example, a skin care device can include a skin-rejuvenation head including a first external region and a second external region, the first external region including an array of skin-cleansing bristles and the second external region including an LED phototherapy appliance adapted to delivery UV-light, visible light, and/or IR-light to a skin surface, wherein the skin-rejuvenation head includes a vibrational motor positioned therein to provide vibratory oscillation to both the skin-cleansing bristles and the auxiliary skin-treatment appliance. In one example, the handle can be configured to dampen vibrational energy introduced at the skin-rejuvenation head, as described previously. The visible light may be in the form of blue-light therapy and/or red-light therapy, for example. The LED phototherapy appliance can be adapted to selectively deliver multiple types of light to the skin surface, such as two or more types of light selected from UV-light, IR-light, blue-light, or red-light, either at the same time or by alternating frequencies.

It is noted that when discussing the various skin care devices herein, and any kits, systems, methods, etc., related thereto, these various more specific discussions can be considered applicable to one another whether or not they are explicitly discussed in the context of that example. Thus, for example, when discussing the vibrational motor in the context of one example, such disclosure is also relevant to and directly supported (but not limiting) in the context of other examples, and vice versa. It is also understood that terms used herein will take on their ordinary meaning in the relevant technical field unless specified otherwise. In some instances, there are terms defined more specifically throughout the specification or included at the end of the present specification, and thus, these terms have a meaning as described herein.

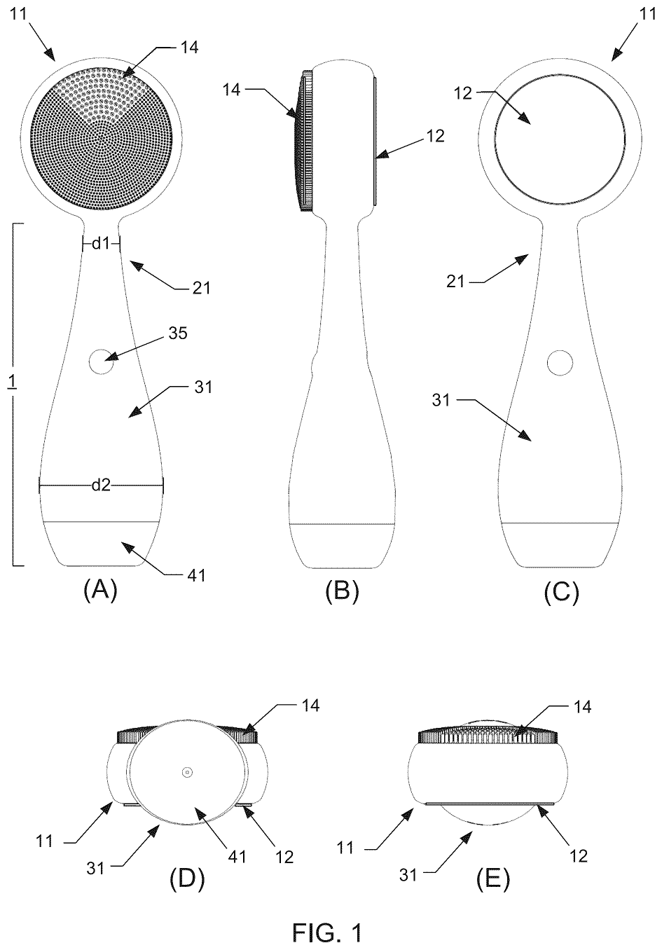

Turning now to one example, as shown in FIG. 1 with five (5) different views, e.g., front view (A), side view (B), back view (C), bottom view (D), and top view (E), a skin care device can include a handle 1 and skin-rejuvenation head 11. The handle in this example includes a neck portion 21 and a grip portion 31. In this example, as shown, the head includes a first external region with an array of skin-cleansing bristles 14 and the second external region with an auxiliary skin-treatment appliance 12, such as a heating pad or other appliance, of a different material and type than the skin-cleansing bristles. A power button 35 and an end cap 41 are also shown.

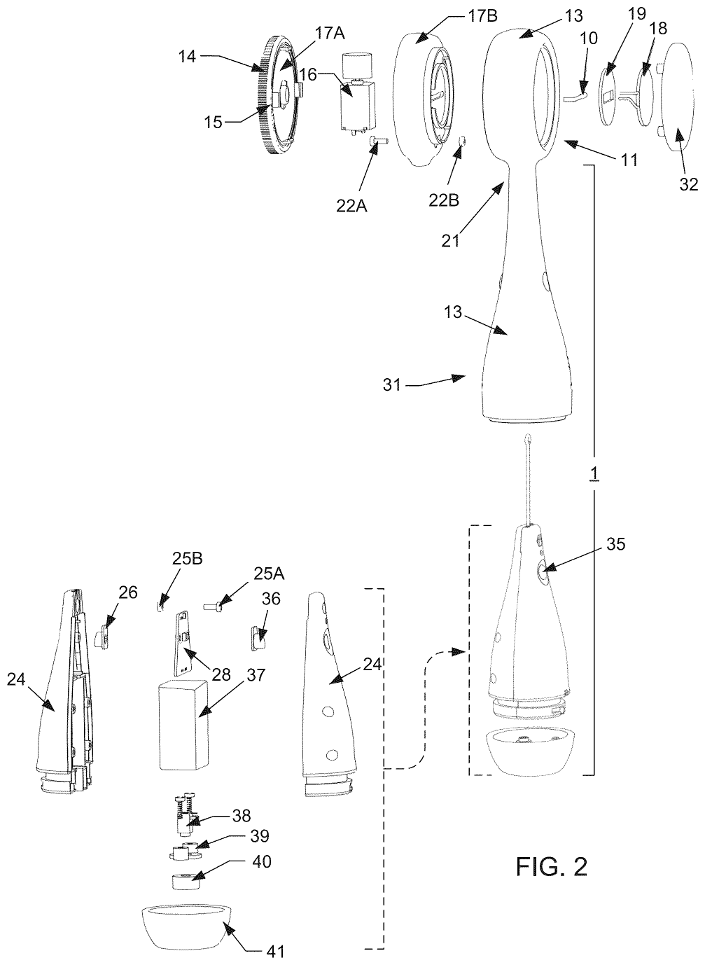

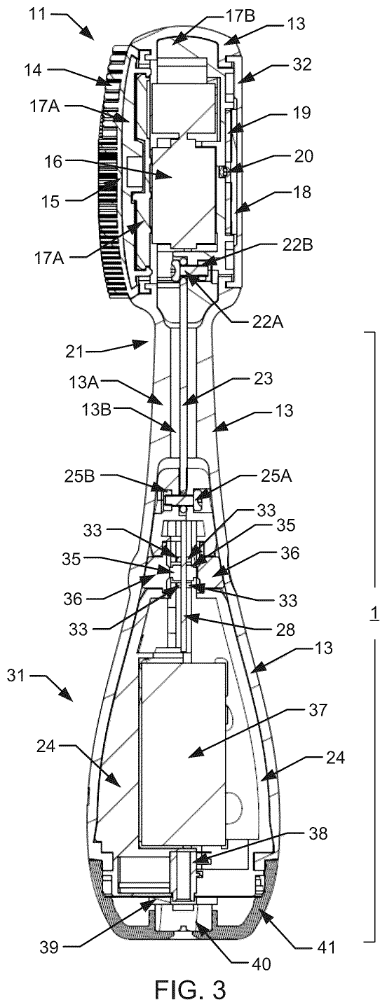

In further detail regarding skin-rejuvenation head 11, as mentioned, a first external region can include an array of skin-cleansing bristles 14 and a second external region can include an auxiliary skin-treatment appliance 12 of a different material and type than the skin-cleansing bristles. The skin-rejuvenation head can also include a vibrational motor (not shown in FIG. 1, but shown in FIGS. 2 and 3) to provide vibratory oscillation to both the skin-cleansing bristles and the auxiliary skin-treatment appliance.

The skin-cleansing bristles can have a length of from 1/16 inch to ⅜ inch, from ⅛ inch to ¼ inch, from 1/16 inch to ¼ inch, or from ⅛ inch to ⅜ inch, for example. The bristles can have a uniform density at the first external region, or can be arranged in multiple sub-regions within the first external region with corresponding different bristle densities, as shown in FIG. 1 (with a top fan-shaped region having a lower bristle density than the rest of the circular shaped “first external region”). An average bristle density at the first external region can be from 100 to 600, from 150 to 500, from 200 to 400 or from 250 to 400 bristles per square inch, regardless if there are multiple sub-regions with different densities. In further detail, however, if the first external region of bristles includes two discrete sub-regions of bristles, then the first sub-region can have a bristle density from 150 to 700, from 200 to 600, from 200 to 500, or from 250 to 400 bristles per square inch, and furthermore, the second sub-region can have a bristle density from 40 to 300, from 50 to 250, from 75 to 250, or from 100 to 200 bristles per square inch, for example. Typically, the second sub-region in this example will have a lower bristle density than the first sub-region. The skin-cleansing bristles at the first external region can have a skin-contact surface area of 2 square inches to 35 square inches, from 3 square inches to 30 square inches, from 3 square inches to 25 square inches, from 3.5 square inches to 20 square inches, from 3.5 square inches to 10 square inches, from 5 square inches to 35 square inches, or from 10 square inches to 35 square inches, for example.

In further detail, the skin-cleansing bristles can be constructed of a rubber material, such as a polysiloxane or a silicone rubber. For example, the silicone rubber that can be hypoallergenic, e.g., medical grade, ultra-hypoallergenic, an ultra-hygienic, and/or odor-resistant silicone rubber. Silicone rubbers are an elastomer of silicone, carbon, hydrogen, and oxygen. Silicone rubbers can include fillers to modulate material properties and/or cost. If fillers are added, they can be added keeping in mind the use as a skin-cleansing brush, and can be selected to be hypoallergenic, hygienic, etc. With this particular type of brush, e.g., a polysiloxanes or silicone rubber bush, the brush can be constructed so that it does not need to be replaced. That stated, it can also be constructed to be modular for periodic replacement, if desired. With these types of silicone rubbers, it can be gentle enough to use on nearly all types of skin, particularly healthy, unbroken skin. In some instances, it can even be gentle enough to use on damaged or diseased skin. The skin-cleansing bristles can be constructed of either the same material or a different material than used as a sleeve over the handle and/or portions of the skin-rejuvenation head. Though silicone rubber can be used effectively, it is noted that other types of bristles brushes can be used as well, depending on the desired skin application and combination with the auxiliary skin-treatment appliance.

The auxiliary skin-treatment appliance, shown generally at 12 in FIG. 1, but shown alternatively with different types of appliances at 32, 42, 52, 62, 72, and 82 by way of example. The auxiliary skin-treatment appliance can be any of a number of sizes at the second external region, for example having a skin-contact surface area of 2 to square inches to 35 square inches, from 3.5 square inches to 30 square inches, from 5 square inches to 25 square inches, or from 10 square inches to 35 square inches, for example. Further detail regarding the auxiliary skin-treatment appliances are described in greater detail hereinafter in the context of FIGS. 2 to 7B.

In this particular example with respect to the handle 1, note that the neck portion 21 includes a location that is narrower (shown at d1) than at a location of grip portion 31 (shown at d2). The diameter (converted to radius; r=d/2) in this example can be used to calculate the cross-sectional areas of the two respective locations. It is noted that if the cross-sectional areas are not defined by a round geometry (perpendicular to the axis of the handle), then other mathematical calculations can be used to determine the respective cross-sectional areas. In this example, as can be seen, the much narrower cross-sectional area (d1) at the neck portion (at least at one location) can be from 4 to 15 times smaller, from 6 to 12 times smaller, or from 8 to 10 times smaller, than a cross-sectional area (d2) of the grip portion (at least at one location). In this specific example, based on the relative diameters shown, the cross-sectional area at (d1) can be calculated to be from about 8 to 10 times smaller than at (d2).

Turning now to FIGS. 2 and 3, an exploded view and a cross-sectional view, respectively, are shown which illustrate aspects of the skin care device of the present disclosure. These two FIGS. will be described together, as they both show different perspectives of the same individual components that can be present in an example device. The example shown in FIGS. 2 and 3 is drawn to embodiments where the auxiliary skin-treatment appliance (shown and described generally in FIG. 1 at 12) is a heating pad 32, such as a metal heating pad. However, as explained in more detail herein, the auxiliary skin-treatment appliance can be other types of appliances. For example, as mentioned, the second external region (separate and distinct from the first external region where the skin-cleansing bristles 14 are located) can include the auxiliary skin-treatment appliance of a different material and type than the skin-cleansing bristles. In other words, the auxiliary skin-treatment appliance is not simply another area with the same material with a different configuration for the same purpose. Rather, it is a different appliance that is attached to the skin-rejuvenation head, typically of a material such as metal, stone, plastic, glass, ceramic, particulates, soft natural material, e.g., loofah, sponge, etc., soft synthetic material (other than the rubber used to form the bristles), etc. For example, the auxiliary skin-treatment appliance can be a thermal pad, e.g., heating pad and/or a cooling pad, an exfoliator, a secondary scrubber (of a different type and material such as a soft porous scrubber), an electromagnetic energy appliance, e.g., phototherapy and/or radio frequency, an ionic infusion appliance, e.g., microcurrent, a massaging appliance, etc. More specific examples include metal heating pads of any metal or metal alloy material; metal heating pads including silver, gold, copper or alloy thereof or a plated substrate thereof, e.g., electroplating on plastic or other non-metallic surface and/or metal on metal plating; semi-precious heating and/or cooling pads, e.g., quartz (such as rose quartz) or jade; LED phototherapy appliances, e.g., red-light, blue-light, etc.; RF frequency skin tightening appliances; skin exfoliation pads or stones, e.g., particulates or crystals embedded in substrates, porous stones, etc.; soft porous scrubbers, e.g., loofahs, sponges, poufs, meshes, etc.; anti-microbial soft porous scrubbers; massaging appliances with massaging relief features; or the like.

In further detail, the skin-rejuvenation head 11 can include a head support shell, shown generally at 17, or more particularly by example in two parts as 17A and 17B, and can be snapped together or otherwise joined, or can be a single part formed around or providing an opening to allow for placement of the vibrational motor 16 that is powered by either direct current (DC) or alternating current (AC). For example, there is a battery 37 present in the handle 1 (within the grip portion 31 in this example) that can power the vibrational motor, though the battery could be placed anywhere that is practical. In this example, the vibrational motor is positioned between the skin-cleansing bristles 14 and the auxiliary skin-treatment appliance 12, shown as a heating pad in this example, but could be any of the auxiliary skin-treatment appliances describe herein. Thus, when in operation, the vibrational motor can deliver vibratory oscillation to both the skin-cleansing bristles and the auxiliary skin-treatment appliance, e.g., heating pad. The head support shell can provide housing and support to the vibrational motor, the skin-cleansing bristles, and the auxiliary skin-treatment appliance, either directly or indirectly through other structures. For example, the skin-cleansing bristles can be supported by a bristle support plate 15 that connects to the head support shell. Likewise, the heating pad can be supported by a heat conductor support 19 connected to the head support shell. The head support shell can also act to provide support directly to the skin-cleansing bristles and the heating pad, in some examples. The heat conductor support can provide support to a heat conductor 18, which can be thermally coupled to the heating pad to transfer an appropriate level of heat to the heating pad.

The heat conductor 18 can be powered by either direct current (DC) or alternating current (AC), but can also be powered by a battery 37. The heat conductor can be a resistive heating element or other type of heating element that can deliver heat to the heating pad, such as a flat and/or low profile coil heater, or a ceramic heating element. The heat of the heat conductor can be such that it delivers a therapeutic temperature to a surface of the heating pad that contacts the skin surface, e.g., from 36° ° C. to 44° C. or from 38° C. to 42° C. If the heating pad is a good heat conductor, like metal, then the temperature of the heat conductor can be about the same as the target temperature for skin contact by the heating pad. If the heating pad is not a good conductor of heat, such as a semi-precious stone, then the temperature at the heat conductor can be higher so that the surface of the semi-precious stone reaches the target skin therapeutic temperature, e.g., from 36° C. to 44° C. or from 38° C. to 42° C. A temperature sensor 20 can also be present to monitor and/or help control the temperature of the heat conductor and/or heating pad 32.

The skin-rejuvenation head 11 can be connected to a handle 1 using a support rod 23, for example. The support rod can be flexible, but rigid enough to provide support between the grasping portion of the handle and the skin-rejuvenation head. The support rod in certain more specific examples can be a solid metal rod, a tube-like rod, or a spring-like rod. The support rod can have any cross-sectional shape, such as circular cross-section, a square cross-section, a rectangular cross-section, a triangular cross-section, a pentagonal cross-section, a hexagonal cross-section, and so forth. The support rod can be metal, polymer, e.g., plastic, rubber, etc., ceramic, etc. As mentioned, the cross-sectional configuration can be solid, tubular, or a combination of both. As mentioned, the handle can include a neck portion 21 and a grip portion 31. In this example, the support rod is mechanically fastened to the skin-rejuvenation head at the head support shell 17A, 17B, adjacent to the vibrational motor 16, using a support rod head coupler 22A, 22B, which in this instance includes a screw and a nut, respectively, but could be any coupler available to affix the support rod to the skin-rejuvenation head. The support rod is also affixed to the handle by a support rod handle coupler 25A, 25B, which is also a screw and a nut, respectively, but could be any other coupling mechanism available to rigidly or semi-rigidly couple the skin-rejuvenation head to the handle. In this example, the coupling occurs at around a junction where the neck portion of the handle meets the grip portion of the handle. However, whether present in the neck portion or the handle region, there is at least a portion of the neck portion between this coupling location (where the support rod is coupled to the handle) and the skin-rejuvenation head.

Also within the handle 1, particularly within the grip portion 31 of the handle in this example, are power and control mechanisms, such as the battery 37, e.g., polymer or other rechargeable battery; a printed circuit board (PCB) 28, e.g., to control the vibrational motor, charging, indicator lights, etc.; a charging port 38; a power button 35, e.g., single vibratory oscillation speed or variable vibratory oscillation speeds; indicator lights 33; and/or various support structures to provide support to these power and control mechanisms, e.g., handle support shell 24 (shown in two pieces in FIG. 2), charging port support 39, power button support 36, etc.

The skin care device can also include an energy dampening sleeve 13 that is positioned as a “skin” over the device, covering a portion of the skin-rejuvenation head (except for the skin-cleansing bristles and the auxiliary skin-treatment appliance, e.g., heating pad or other appliance, in this example), most of the handle 1, e.g., the neck portion 21, and most of the grip portion 31. The energy dampening sleeve can be constructed of a polysiloxanes or silicone rubber, for example. At a bottom end of the handle, in this example, there is an end cap 41, which is associated with a charging port opening 40. The end cap can be rigid, for example, and the charging port opening can be rubber to provide a water-tight or near water-tight seal, where a charging cable (not shown) can be passed through to become inserted into the charging port 38. With more specific reference to the energy dampening sleeve, notably, at the neck portion, where the support rod is unconnected to either the skin-rejuvenation head or the handle, there is a thickened portion 13A of the energy dampening sleeve 13. The thickened portion of sleeve, e.g., silicone or other rubber sleeve, in this example provides a gap 13B. It is noted that the energy dampening sleeve does not need to extend to the skin-rejuvenation head 11 or the grip portion 31 go be effective at dampening energy; but in some examples, the energy dampening sleeve is a continuous polymeric or rubber sleeve that spans from the grip portion onto the skin-rejuvenation head. Furthermore, as mentioned previously, the neck portion includes one or more locations where its cross-sectional area can be from 4 to 15 times smaller (or from 6 to 12 times smaller, or from 8 to 10 times smaller) than the cross-sectional area of at least one location in the grip portion. This combination of (i) having a support rod coupling and providing support between the handle and the skin-rejuvenation head, (ii) having a thickened portion of the energy dampening sleeve along the neck portion (compared to other areas such as may be present at the skin-rejuvenation head, and/or (iii) having a gap (or space) about the support rod between the thickened portion of the energy dampening sleeve and the support rod, transfer of vibrational energy from the skin-rejuvenation head to the grip portion of the handle can be reduced over other structures that do not have these mechanical vibration dampening features. Using one or two of these design features can likewise dissipate energy from the vibration of the head (relative to the grasping portion), but using two or three of these design features can provided added anti-vibrational properties. This vibrational dampening can be helpful with this type of device, as the presence of particularly the auxiliary skin-treatment appliance and associated structures that may be present to operate this appliance can add enough weight to the skin-rejuvenation head so as to make vibrational transference more noticeable to users. Furthermore, this can be particularly the case when the vibrational motor is potentially delivering a vibrational oscillation frequency from 7,000 vibrations per minute (VPM) to 12,000 VPM. With that stated, example VPM frequencies that can be implemented at a fixed frequency, or with multiple fixed frequency settings, e.g., 2 discrete settings, 3 discrete settings, 4 discrete settings, etc., or with variable frequency settings, e.g., frequencies dialed up and down as desired, can be from 2,000 VPM to 12,000 VPM, 4,000 VPM to 12,000 VPM, from 6,000, VPM to 12,000 VPM, from 7,000 VPM to 12,000 VPM, from 2,000 VPM to 10,000 VPM, 4,000 VPM to 10,000 VPM, from 6,000, VPM to 10,000 VPM, from 7,000 VPM to 10,000 VPM, from 2,000 VPM to 8,000 VPM, 4,000 VPM to 8,000 VPM, from 6,000, VPM to 8,000 VPM, from 7,000 VPM to 8,000 VPM, from 2,000 VPM to 6,000 VPM, from 4,000 VPM to 6,000 VPM, from 6,000 VPM to 10,000 VPM, from 7,000 VPM to 9,000 VPM, from 7.000 VPM to 8,000 VPM, or from 8,000 VPM to 12,000 VPM

In one example, from 7,000 VPM or more, e.g., 7,000 VPM to 12,000 VPM with respect to the skin-cleansing bristles, a deep cleaning can occur. With respect to either the first external region (skin-cleansing bristles) or the second external region (auxiliary skin-treatment appliance), these vibration energies can provide a way to lift, firm, and tone problem areas of the skin. For example, at 7,000+ VPM, the skin-cleansing bristles can assist with removing skin impurities and breaking down dirt and oil from within the pores. At these vibrational speeds, the bristles and/or the heating pad can act to deeply massage serums or moisturizer into the skin. Heat treatment can also provide the benefit of opening pores, promoting improved circulation, promoting skin care fluids to transdermally pass into or across the epidermis, relaxing underlying muscles, providing moist heat when used with a skin care fluid which may penetrate deeper than dry heat and may hydrate the skin, releasing acne-causing bacteria from skin pores, and/or other similar benefits. With some of the benefits associated with application of heat, particularly with respect to skin treatment, cleansing using the array of skin-cleansing bristles described herein can further contribute to skin health. For example, cleansing prior to application of heat can improve absorption of skin treatment fluids or even transdermal (into or even through the epidermis) transfer of some skin care fluids, e.g., moisturizer, serums, etc.

As shown in FIGS. 2 and 3, the auxiliary skin-treatment appliance can be a heating pad 32, such as a metal heating pad, as shown. However, instead of (or in addition to) a heat conductor 18, cooling architecture or components can be included to thermally transfer cooling temperatures for cooling applications. Thus, in some examples, the “heating pad” can be referred to as a thermal skin-treatment pad, e.g., for heating and/or cooling. In this example, the thermal skin-treatment pad can be any metal or other material suitable for transferring thermal treatment properties to the skin surface. In one example, the thermal skin-treatment pad can be any thermally conductive material suitable for providing heat or cooling to the skin surface, without damaging the skin. For example, the thermal skin-treatment pad can be associated with a heat conductor to provide heat to a skin surface through the metal skin-treatment pad. Alternatively, the metal skin-treatment pad can be associated with a cooling source to provide cooling to a skin surface through the metal skin-treatment pad. These temperatures (heating and/or cooling) can be used with or without serums and/or moisturizers, for example. With respect to heating, temperatures can be used that will not burn the skin, e.g., 36° ° C. to 44° C. or from 38° C. to 42° C. can be suitable. Temperatures outside of this range can be used but may cause safety concerns. With respect to cooling, temperatures from −10° C. to 20° C., from −5° C. to 15° C., or from 0° ° C. to 15° C., or from 0° ° C. to 10° C. can be used, taking user safety into account, as the mean temperature where frostnip can occur with prolonged exposure may be at about −10° ° C. for many subjects. Thus, temperature and time of application can be considered when using cooling pads in accordance with the present disclosure.

Heating can be provided as described in connection with FIGS. 2 and 3, as mentioned. With respect to cooling architectures that can be used, cooling coils with refrigerant or expanding gas can be used in thermal communication with the thermal skin-treatment pad, for example. In one example, an electrocaloric cooling device or a thermoelectric cooler can be used. As an illustration, a polymer layered between a heat sink and a heat source can generate cooling when electric current is applied to the polymer touching the heat sink. The polymer may, for example, be such that the polymer molecules become aligned to some degree, forcing heat into the heat sink, and then when the electrical current is turned off and the molecules relax, the temperature drops. Repetition of this type can lead to cooling using a small device that could be included in a hand-held device as described herein. There are also thermal chemical reactions that can be leveraged to generate cooling. In another example, cooling can be provided by ammonium nitrate in water. For example, a particulate ammonium nitrate can be reacted with water to generate water cooling, which can be circulated to the thermal skin-treatment pad to provide cooling thereto. Alternatively, the cooling reaction chamber can be located just beneath the thermal skin-treatment pad. Either way, when the ammonium nitrate has been spent, the device can include a mechanism for replacement of the reactant, such as by using a cartridge or other similar mechanism.

Whether heating or cooling, if a metal thermal skin-treatment pad is used, in one example, it can be constructed to include a metal selected from silver, gold, copper, or an alloy thereof. The thermal skin-treatment pad can be, in one example, plated with the metal or metal alloy, e.g., silver-plated, gold-plated, copper-plated, or plated with an alloy of silver, gold and/or copper. These metals can be used for multiple purposes, including anti-microbial treatment of the skin and/or delivery of metal ions to the surface of the skin in some examples. The metals can be in the form of elemental metals, or can be alloys, as mentioned. The alloys can include combinations of silver, gold, and/or copper, but can also include other elements, such as multivalent metals, e.g., e.g., zinc, iron, calcium, magnesium, manganese, chromium, etc.; monovalent metals, e.g., potassium, sodium; dopant such as alloys with silicon, oxygen, sulfur, phosphorus, etc.; and/or antioxidant compounds.

If a semi-precious stone skin-treatment pad is used, stone pads constructed from quartz and/or jade, for example, can be used. If quartz is used, in one specific example, the quartz can be rose quartz, which is pink in color and can display diasterism or star-like concentrations of reflective and/or refractive properties. The color of rose quartz may often be due to the presence of trace amounts of titanium, iron, and/or manganese embedded or formed within the crystalline structure of the quartz material, but may also be due to the microscopic fibers of dumortierite, which is a variably colored aluminum boro-silicate material. The pink color may be due to the substitution of some iron and/or other trivalent metals for some of the aluminum. The presence of some of these metals, and other elements and/or complexes in rose quartz, may be the reason rose quartz can provide a therapeutic effect when used on the surface of skin. In another example, there is also a type of pink quartz, sometimes called crystalline rose quartz, which can be used, though this material is rarer than rose quartz. The pink color may be caused by trace concentrations of phosphate or aluminum. Both types of rose quartz, e.g., rose quartz and crystalline rose quartz, are considered to be “rose quartz” in accordance with the present disclosure. In further detail, rose quartz includes many different metals and minerals that can assist with healthy skin, including silicium, magnesium, iron, sodium, and oxygen. Rose quartz can also reduce inflammation as well as provide improved cellular renewal and rejuvenation. The presence of oxygen rose quarts can act to enhance circulation and improve skin tone, luster, and glow. Other types of quartz that can be used for the thermal skin-treatment pad include colorless quartz crystals, such as Herkimer diamond or rock crystal; amethysts (rich in iron); amentrines; chalcedonies, such as agate, jasper, onyx, etc., carnelian, milky, smoky, tiger's eye, citrine, prasiolite; dumortierite; aventurine; or the like. These other types of quartz bring different levels of metals and other minerals to the skin surface, and can be used with similar effectiveness in many instances.

If jade is used, then any of the various types of jade can be selected for use, including nephrite, jadeite, and/or xiuyan jade semi-precious stones. There are more types of jades than these three green-types of jade stones, e.g., white jade. Jade can be used with heat and/or cooling to help improve skin elasticity, encourage lymphatic drainage, and/or help prevent wrinkles. Jade can also be used to introduce metals in trace amounts to the skin surface. The metal content of jade can include magnesium and calcium, which can be beneficial for treating the skin in accordance with examples of the present disclosure. The metal content of nephrite, for example, includes calcium, magnesium, and iron. These elements can be present in the form of the magnesium-iron rich amphibole mineral series tremolite, e.g., (calcium-magnesium)-ferroactinolite (calcium-magnesium-iron). One example chemical formulation for nephrite-type jade is Ca₂Mg₅(OH)₂(Si₄O₁₁)₂. Thus, like quartz, there can be a considerable concentration of silicon and oxygen. However, nephrite also includes relatively large concentrations of calcium and magnesium, as well as iron. These metals can, in some instances, be beneficial to skin treatment as described herein. Jadeite, on the other hand, is rarer than nephrite and often more desirable for use with jewellery, but in the context of the present disclosure, may not deliver as much metal content to the skin during use. That stated, it can still be used effectively. Jadeite is sometimes referred to as “elemental stone,” which is a misnomer because it is made up of several elements. Jadeite is a pyroxene with metal content including sodium, aluminum, and iron. An example general formula for jadeite is Na(Al,Fe³⁺)Si₂O₆. Thus, jadeite can be useful for delivery of particularly trivalent iron to the skin surface, along with sodium and aluminum. Xiuyan, on the other hand, is often rich in serpentine formations of metal and other mineral content, and can also include a particularly high concentration of magnesium, with a relatively small concentration of calcium, e.g., MgO at about 40 wt % to 44 wt %, SiO at about 42 wt % to about 45 wt %, Ca) at about 0.13 wt %. If the intent is to use a jade with minimal metal interaction with the skin surface, then white jade can be selected for use, as it typically includes lower metal content than some of the green jades previously described. Regardless of the jade chosen for use, the application of heat and or cooling to the jade can provide beneficial properties to the skin surface, and can be used to conduct thermal heating and/or cooling as well as to introduce serums, moisturizers, or other fluids to the skin surface. For example, jade can have the effect of flattening wrinkles and assist with reducing inflammation by clearing fluid congestion, e.g., lymphatic drainage. When used cold, it can help tighten pores and skin. Jade applied to the skin as described herein can also act to tone up facial muscles, increase circulation, and aid in the absorption of serums and/or moisturizers in some instances.

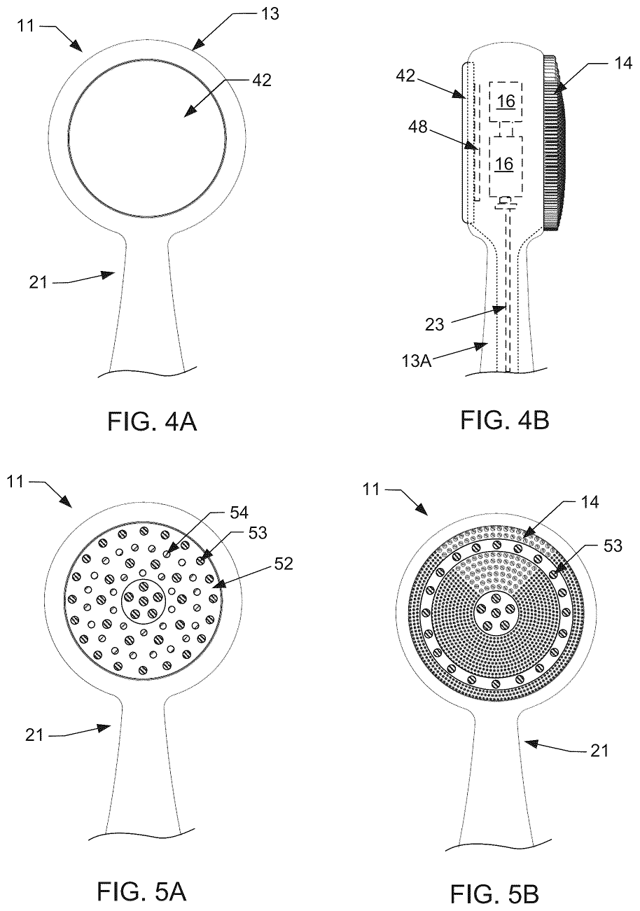

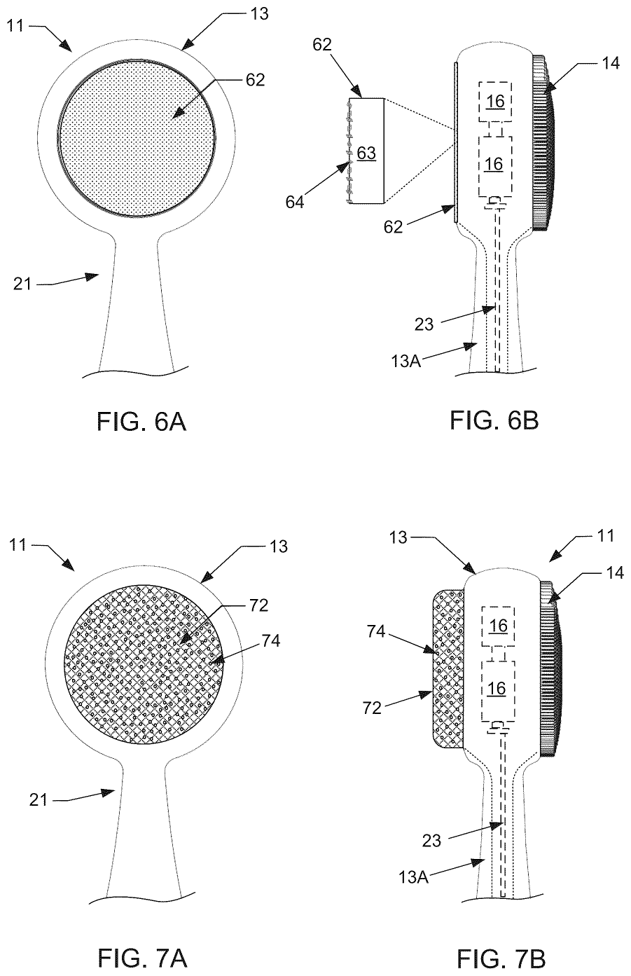

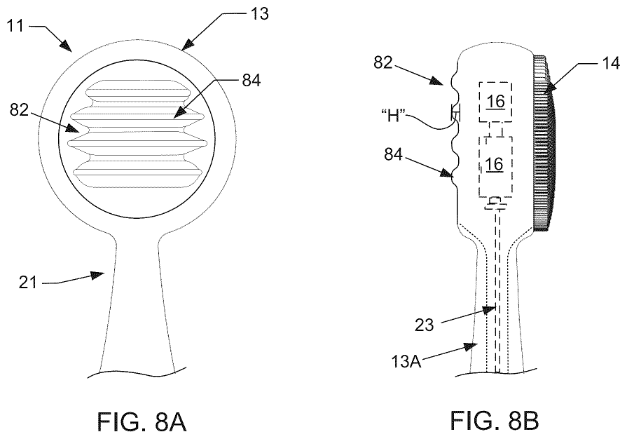

In other examples, there are auxiliary skin-treatment appliances that can be used that deliver treatments to the skin other than heat. Examples include ionic infusion therapy appliances such as that shown in FIGS. 4A and 4B, phototherapy appliances such as that shown in FIGS. 5A and 5B, radio frequency (RF) therapy appliances (not shown, but can be similar in configuration as that shown in FIGS. 5A and 5B but with RF emitters rather than LED phototherapy emitters), exfoliation therapy appliances such as a pumice stone or the microdermabrasion appliance shown in FIGS. 6A and 6B or the soft porous scrubber with an anti-microbial metal embedded or infused therein as shown in FIGS. 7A and 7B, massage therapy appliances for massaging the skin and underlying muscles as well as to provide a surface for working skin care fluids onto or into the skin as shown in FIGS. 8A and 8B. These various appliances are described in further detail hereinafter.

As shown in FIGS. 4A and 4B, the skin care device can, in some specific examples, include some of the same features described previously with respect to the skin-rejuvenation head 11, the neck portion 21, the grip portion (not shown), the energy dampening sleeve 13 including the thickened portion 13A, the support rod 23, the vibrational motor 16, the skin-cleansing bristles 14, etc. However, in this example, rather than a heating pad or a cooling pad, the auxiliary skin-treatment appliance can be an ionic infusion appliance 42 that conducts electrical current. The ionic infusion appliance can deliver a microcurrent, for example, from the same battery (not shown in these FIGS.) or power source that operates the vibrational motor, or by a separate power source or supply. The printed circuit board (not shown in these FIGS.) can thus be used to control the microcurrent applied to the ionic infusion appliance, or a separate control device can be used to control the microcurrent (separate from operation of the vibrational motor). In this example, there are microcurrent electrodes 48 that can be used to receive and transfer the microcurrent to the ionic infusion appliance. These electrodes are shown as a single pair of electrodes that are spaced apart at either end of the ionic infusion appliance (in cross-section), but this is exemplary only, as the electrodes could be in the form of any number of electrodes in any number of configurations suitable for delivering the microcurrent to the conductive ionic infusion appliance, e.g., interdigitated finger electrodes, concentric ring electrodes, etc.

Microcurrent therapy, or ionic infusion therapy, can be used to delivery microcurrent to the skin of a user, using electrical current at from about 5 HA up to about 1,000 μA (1 mA), from about 10 μA up to about 750 μA, from about 10 μA up to about 500 μA, or from about 50 μA up to about 400 μA, for example. The microcurrent setting can be fixed, or can be provided at various levels, continuous variability or stepped/fixed μA levels. The application of microcurrent can, in some applications, increase adenosine triphosphate (ATP), which is a source of energy synthesized from nutrients during cellular respiration. By increasing ATP at the skin, ion exchange across the cellular membrane can improve nerve conductivity, for example. Microcurrent can also promote fibroblast production, which correlate positively to collagen and elastin levels within the skin. Visible results can include wrinkle reduction (building collagen and elastin though the production of fibroblasts), reduced skin sagging, and in some instances can promote wound healing. Microcurrent can also have a positive impact on muscles underlying the skin, further contributing to skin firmness. In cases where there may be skin inflammation, microcurrent can in some instances promote healing with a reduction in swelling through the stimulation of lymphatic drainage. Microcurrent can also promote cellular uptake of nutrients, for example. Treatment for 3 to 15 minutes every day, twice a week, every week, every two weeks, every month, etc., can provide benefits. In further detail, by combining this type of therapy with the use of the skin-cleansing bristles and vibratory oscillation can provide a good combination of treatment that promotes healthy skin.

Alternatively, the device shown in FIGS. 4A and 4B can be constructed similarly, except that rather than an ionic infusion appliance, auxiliary skin-treatment appliance can be a radio frequency appliance for applying RF energy to a skin surface. RF energy can be used, for example, for skin tightening similar to that described with respect to ionic infusion therapy. The frequency of RF signals applied can be from about 350 kHz to about 550 KHz, or from about 400 kHz to about 500 kHz, for example, though other electromagnetic frequencies can be used as appropriate for a given skin treatment regimen. In one example, skin tightening can be effective with frequencies of about 450 KHz. RF treatment can provide multiple functions, including providing heat to the treated tissues, as well as stimulating subdermal collagen production. This can result in skin tightening and wrinkle reduction, for example. As with ionic infusion, collagen and elastin building can occur, but in addition, RF treatment can cause apoptosis of fat cells, which results in reduced fat in the treated areas. Lax skin can thus be treated on the face, but also on other problematic areas, such as the stomach, thighs, upper arms, jowls, etc.

In FIGS. 5A and 5B, the skin care device can, in some specific examples, include some of the same features described previously with respect to the skin-rejuvenation head 11, the neck portion 21, the grip portion (not shown), the energy dampening sleeve 13 including the thickened portion 13A, the support rod 23, the vibrational motor 16, the skin-cleansing bristles 14, etc. However, in this example, the auxiliary skin-treatment appliance can be a phototherapy appliance 52, such as an LED phototherapy appliance with LED sources 53, 54. The photo therapy appliance can be used for applying UV-light, visible light, or IR-light to a skin surface. There can be one type of LED light source present, or multiple types of light sources present, as shown in FIG. 5A. In some examples, the side of the skin care device with the skin-cleansing bristles may also include LED light sources, shown by example in FIG. 5B. More specific examples of LED light sources that can be used include blue-light sources and/or red-light sources. Blue-light therapy, for example, can be used for treating acne and/or other similar conditions. This is because it can kill bacterial growth at the surface of the skin, and in some instances, just beneath the surface of the skin. Blue-light can also calm inflammation in some instances. Red-light therapy, on the other hand, can penetrate deeper into the skin than the blue-light due to it having a longer light wavelength. By reaching layers deeper in the skin, red-light therapy can reach where collagen and elastin are located at a greater concentration. Thus, red-light therapy can rejuvenate damaged skin with broken or slowed metabolic activity, and can also rejuvenate capillaries, enhancing blood flow, bringing with that enhanced oxygen and nutrient levels. Application of both blue-light and red-light can be carried out simultaneously. Phototherapy can be carried out daily, weekly, bi-monthly, monthly, etc., taking into account the time of treatment, the intensity of light, and the condition being treated.

In another example as shown in FIGS. 6A and 6B, the skin care device can, in some specific examples, include some of the same features described previously with respect to the skin-rejuvenation head 11, the neck portion 21, the grip portion (not shown), the energy dampening sleeve 13 including the thickened portion 13A, the support rod 23, the vibrational motor 16, the skin-cleansing bristles 14, etc. However, in this example, the auxiliary skin-treatment appliance can be an exfoliation appliance 62, such as a porous stone, e.g., pumice stone (now shown), or alternatively, a substrate 63 embedded with particulates 64, such as aluminium oxide particulates or crystals. For example, as shown in this FIG., a polymeric substrate can include aluminium oxide particles (or crystals) that are embedded therein. The embedding can by a thermal process, where a polymeric substrate, e.g., plastic substrate, is warmed to a softening temperature so that the aluminium oxide particles can be pressed or otherwise sink into the softened plastic substrate. As an alternative method, the polymeric substrate, e.g., plastic substrate, can be dipped in a mixture of the aluminum oxide particles and a softening solvent vehicle, such as methyl ethyl ketone (MEK) for a short period of time, e.g., 10 seconds to 2 minutes, 10 seconds to 50 seconds, etc., and then the substrate is removed for hardening. In this example, the MEK softens the plastic of the substrate, allowing aluminum oxide particles to become embedded. After it plastic hardens again they are embedded within the plastic. In preparation of the exfoliation appliance of this type, aluminium oxide crystals can be selected to at a particle size and added at a density to provide an 80 grit to 300 grit, a 150 grit to 250 grit, or an 80 grit to 150 grit exfoliation surface. Notably, the aluminum oxide particles, e.g., crystals, can be present as one of multiple types of particulates included on the exfoliation appliance. For example, the particulates can be a combination of aluminum oxide crystals mixed with a second type(s) of particles.

In another example as shown in FIGS. 7A and 7B, the skin care device can, in some specific examples, include some of the same features described previously with respect to the skin-rejuvenation head 11, the neck portion 21, the grip portion (not shown), the energy dampening sleeve 13 including the thickened portion 13A, the support rod 23, the vibrational motor 16, the skin-cleansing bristles 14, etc. However, in this example, the auxiliary skin-treatment appliance can be a soft porous scrubber 72 selected from a natural loofah, a natural sponge, a synthetic mesh loofah, a synthetic sponge, for example. The soft-porous scrubber can likewise be a pouf or a nylon (or other type) mesh. The nylon mesh, for example, can be configured or bunched in a manner to provide increased cleaning surface are compared to a flat mesh. In one example, the soft porous scrubber can include an anti-microbial metal 74 associated therewith. For example, the anti-microbial metal can include elemental silver, elemental copper, elemental gold, alloys of silver, alloys of copper, alloys of gold, ionic silver, ionic copper, ionic gold, a silver compound, a copper compound, a gold compound, or combinations thereof.

To provide an example, the soft porous scrubber can be a synthetic mesh loofah that can be treated with a silver. The silver can be applied using anti-bacterial solution or dispersion to provide a source of silver ions to the loofah. Example silver sources include colloidal silver (elemental silver particles or colloids, e.g., nanoparticles or microparticles), silver chloride (AgCl₂), silver nitrate (AgNO₃), silver sulfadiazine, silver zeolite (e.g., aluminosilicate with silver ions carried by micropores), etc. Silver and/or other anti-microbial metals can be used in combination as alloys, or as alloys with other metals, oxidizers, or even UV-light for anti-microbial effect with added effectiveness in some examples. In an alternative example, with respect to the natural loofahs, these can be naturally harvested materials used for cleansing the skin. For example, a natural loofah can be harvested naturally from the fibrous interior of a mature loofah fruit that resembles a marrow, and then is dried for uses as a natural sponge for cleaning the body. A loofah (synthetic or natural), or any of the other soft porous scrubbers described herein, can be loaded with an anti-microbial metal, such as by metal or metal compound adsorption on a surface, impregnation into pores or openings, or infused into a material of the soft porous scrubber, etc.

An example of a technology where silver salt is loaded onto or into fabrics/textiles to prevent bacterial odors and to provide other antimicrobial properties is available under the trade name Polygiene®, from Polygiene AB (Sweden). Polygiene® has been used primarily for fabrics associated with sportswear. This type of silver treatment, or any other type of anti-bacterial metal treatment described herein can thus be used to provide antimicrobial activity to the soft porous scrubbers of the present disclosure, including when used as the auxiliary skin-treatment appliance described herein.

In one example where a particulate silver source is used to provide silver ions to the soft porous scrubber, particularly when wetted and in use, the particulate silver source can be one to two orders of magnitude larger than many conventional nanoparticles, e.g., from hundreds of nanometers in size to particles in the micron range, e.g., 250 nm to 10 μm, 500 nm to 10 μm, 750 nm to 10 μm, from 1 μm to 10 μm, from 250 nm to 1 μm, from 500 nm to 5 μm, etc. As larger particles, they may be too large to penetrate the skin, but rather, simply be used as an effective surface antimicrobial ion source.

In another example as shown in FIGS. 8A and 8B, the skin care device can, in some specific examples, include some of the same features described previously with respect to the skin-rejuvenation head 11, the neck portion 21, the grip portion (not shown), the energy dampening sleeve 13 including the thickened portion 13A, the support rod 23, the vibrational motor 16, the skin-cleansing bristles 14, etc. However, in this example, the auxiliary skin-treatment appliance can be a massaging appliance 82 with massaging relief features 84, e.g., ridges, bumps, valleys, divots, or the like. In one example, the massaging appliance (including massaging relief features) can be of the same material as the energy dampening sleeve. For example, the massaging features can be constructed of thickened portions of raised features such as ridges, bumps, etc., or thickened portions can define thinner portions of recessed features such as voids, valleys, divots, etc. These massaging relief features can be formed using the material of the energy dampening sleeve, e.g., part of a continuous energy dampening sleeve that provides the massaging features. In another example, the massaging features can be constructed beneath the energy dampening sleeve, and the sleeve can act to cover the features constructed therebeneath. In another example, the massaging appliance can be of a different material than the energy dampening sleeve, if present at the skin-rejuvenation head. Regardless of how constructed, with raised or recessed features, these features can have a height or depth suitable so that when massaging the skin, skin care fluid, e.g., serums, can collect or become pooled where the massaging appliance does not contact the skin, thus providing a reservoir to draw from during application, thus prolonging the amount of time that massaging with serum or other skin care fluid can occur. Example relief feature height or depth, notated in FIG. 8B as “H.” comparing the highest point to the lowest point on the massaging appliance, can be from about 1/32 inch to about ⅜ inch, from 1/16 inch to ¼ inch, from 1/32 to ⅛ inch, or from ⅛ inch to ⅜ inch, for example.

In accordance with examples of the present disclose, the devices herein can be used as part of a system or kit, where the skin care device is co-packaged or used together with any of a number of skin care fluids, such as serums, essences, ampoules, moisturizers, creams or ointments, topical pharmaceutics, topical nutricosmetics or cosmeceutics, or the like. These types of skin care fluids can carry compounds to the skin, and can include a liquid carrier, e.g., aqueous carrier or oil-based carrier, which carry an active ingredient to (or into) the epidermis. Example oils can include argan oil, rose oil, vitamin oil, seed oil, e.g., pomegranate seed oil, rose hip see oil, etc., jojoba oil, nut oil, e.g., macadamia nut oil, kukui nut oil, etc., oil from fruit or flowers, e.g., apricot kernal oil, orange oil, lemon oil, neroli flower oil, jasmine oil, coconut oil, etc., aloe, hemp oil, or the like. The oil can act as the active ingredient in part, or can be merely a carrier for an active ingredient. Active ingredients, on the other hand, can include metal(s) and other minerals, antioxidant(s), fatty acids, vitamins, e.g., vitamin E, vitamin C, vitamin A, etc., drugs, other anti-aging compounds, anti-inflammatory agents, e.g., aloe, green tea, etc., organic acids, e.g., mandelic acid, malic acid, hyaluronic acid, salicylic acid, etc., skin-brightening compounds, anti-acne compounds, e.g., salicylic acid, antimicrobial compounds, e.g., colloidal or ionic silver compounds, hydrating compounds, proteins, peptides, amino acids, amino acid chelates or other chelates, or the like. In one example, the skin care devices described herein can include an anti-aging or an anti-wrinkle serum, such as a serum with mandelic acid and/or malic acid. In another example, the serum can include a peptide.

In accordance with another example, a method of skin rejuvenation can include using any of the devices described herein for cleansing the skin using the skin-cleansing bristles with a solvent, such as water or other solvent, and optionally a surfactant or other skin cleanser. The various methods can further include, depending on which device is being used, treating the skin using the auxiliary skin-treatment appliance. The step of treating the skin can include, for example, using the auxiliary skin-treatment appliances found on the same skin-rejuvenation head as the skin cleansing bristles (used in the first step). Examples of using such an appliance may include heating and/or cooling the skin using a metal thermal skin-treatment pad, e.g., metal or metal alloy or plated metal or plated metal alloy which may include silver, gold, copper, etc. Another example may include heating and/or cooling the skin using a semi-precious stone pad as described on the skin care devices herein, e.g., quartz skin-treatment pad and/or jade skin-treatment pad, etc. Using the auxiliary skin-treatment appliance may likewise include working serums or other skin care fluids into the skin using the appliance (with or without heat and/or cooling, e.g., metal or semi-precious stone pads); exfoliating the skin using an exfoliation pad described herein; treating the skin with phototherapy using a phototherapy appliance described herein, e.g., blue-light, red-light, alternating blue-light and red-light, simultaneous blue-light and red-light, UV-light (such as for antimicrobial treatment), IR-light (such as for deep skin penetration), etc; treating the skin with RF energy at frequencies to promote skin tightening; scrubbing the skin with one of the soft porous scrubbers (with or without the antimicrobial metal); etc. Other methods may include using an auxiliary skin-treatment appliance to transfer beneficial metal and/or minerals to or into the skin surface. For example, metal ions can be transferred to the skin surface using metal skin-treatment pads, e.g., ionic silver or other metal transfer from metal skin treatment pads with or without the use of serums or other fluids, metal or mineral transfer from mineral rich quartz or jade skin-treatment stone pads, e.g., rose quartz or nephrite jade, etc. These methods can be implemented with vibrational energy to one or both steps, namely the step of cleansing the skin using the skin-cleansing bristles and/or the step of treating the skin using the auxiliary skin-treatment appliance (of any of the types described herein). Vibratory oscillation can be at any frequency described herein, but in one example, can be at least 7,000 VPM, from 7,000 VPM to 12,000 VPM, or from 8,000 VPM to 10,000 VPM.

It is noted that, as used in this specification and the appended claims, the singular forms “a,” “an,” and “the” include plural referents unless the context clearly dictates otherwise. In describing and claiming the teachings of the present disclosure, the following terminology will be used in accordance with the definitions set forth below.

Dimension, amounts, concentrations, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a numerical range of “about 10 to about 50” should be interpreted to include not only the explicitly recited values of about 10 to about 50, but also to include individual values and sub-ranges within the indicated range. Thus, included in this numerical range are individual values such as 20, 30, and 40 and sub-ranges such as from 10-30, from 20-40, and from 30-50, etc. This same principle applies to ranges reciting only one numerical value. Furthermore, such an interpretation should apply regardless of the breadth of the range or the characteristics being described.

It is to be understood that the above-referenced arrangements are only illustrative of the application of the principles of the present disclosure. Numerous modifications and alternative arrangements can be devised without departing from the present disclosure. While the present disclosure has been shown in the drawings and fully described above with particularity and detail in connection with what is presently deemed to be the most practical embodiment(s) of the disclosure, it will be apparent to those of ordinary skill in the art that numerous modifications can be made without departing from the principles and concepts of the disclosure as set forth herein.

What is claimed is:

1. A skin care device, comprising a skin-rejuvenation head including a first external region and a second external region, the first external region including an array of skin-cleansing bristles and the second external region including an LED phototherapy appliance adapted to delivery UV-light, visible light, or IR-light to a skin surface, wherein the skin-rejuvenation head includes a vibrational motor positioned therein to provide vibratory oscillation to both the skin-cleansing bristles and the auxiliary skin-treatment appliance.

2. The skin care device of claim 1, further comprising a support rod mechanically coupled to the skin-rejuvenation head.

3. The skin care device of claim 2, wherein the support rod includes a rigid spring, a flexible metal, or a plastic rod.

4. The skin care device of claim 2, further comprising a handle including a neck portion and a grip portion, wherein the support rod coupled to the handle, and wherein the neck portion is proximal to the skin-rejuvenation head and the grip portion is distal to the skin-rejuvenation head, the neck portion including an energy dampening sleeve that surrounds the support rod, leaving a gap around the support rod.

5. The skin care device of claim 4, wherein the energy dampening sleeve surrounds the support rod and leave an air gap around the support rod within the neck portion, the support rod connecting to the handle within the grip portion.

6. The skin care device of claim 5, wherein the energy dampening sleeve does not provide enough structural support to support the skin-rejuvenation head in an upright position relative to the handle without the presence of the support rod.

7. The skin care device of claim 4, wherein the skin care device includes a rubber sleeve that covers at least part of the grip portion, the neck portion, and at least part of the skin-rejuvenation head, wherein the energy dampening sleeve at the neck portion is part of the rubber sleeve, and wherein the energy dampening sleeve has an average outer wall thickness that is greater than the average outer wall thickness of rubber sleeve at the skin-rejuvenation head, the grip portion, or both.

8. The skin care device of claim 4, wherein the neck portion includes a narrow a cross-sectional area that is from 4 to 15 times smaller in area than at least one a cross-sectional area of the grip portion.

9. The skin care device of claim 1, wherein the LED phototherapy appliance is adapted to deliver UV-light to the skin surface.

10. The skin care device of claim 1, wherein the LED phototherapy appliance is adapted to deliver visible light to the skin surface.

11. The skin care device of claim 1, wherein the LED phototherapy appliance is adapted to deliver IR-light to the skin surface.

12. The skin care device of claim 1, wherein the LED phototherapy appliance is adapted to deliver red-light to the skin surface.

13. The skin care device of claim 1, wherein the LED phototherapy appliance is adapted to deliver blue-light to the skin surface.

14. The skin care device of claim 1, wherein the LED phototherapy appliance includes multiple types of light sources to delivery multiple types of light to the skin surface.

15. The skin care device of claim 14, wherein the multiple types of light sources are adapted to deliver two or more of types of light selected from UV-light, IR-light, blue-light, or red-light.

16. The skin care device of claim 1, wherein the skin-cleansing bristles are constructed from a silicone rubber or a polysiloxane, and the skin-cleansing bristles are hypoallergenic.

17. The skin care device of claim 1, wherein the skin-cleansing bristles are from 1/16 inch to ⅜ inch in length on average, and are present on the head at a density from 100 bristles per square inch to 500 bristles per square inch, and wherein the skin-cleansing bristles at the first external region have a skin-contact surface area of 2 square inches to 35 square inches.

18. The skin care device of claim 17, wherein the skin-contact surface area is from 3 square inches to 25 square inches.

19. The skin care device of claim 1, wherein the skin-cleansing bristles include two discrete locations of bristles, wherein the first external region includes bristles at a first bristle density and a second external region includes bristles at a second bristle density.

20. The skin care device of claim 1, wherein the first external region also includes LED phototherapy source lights.

21. The skin care device of claim 1, wherein the vibrational motor is oscillatable at one or more setting from 7,000 VPM to 12,000 VPM.

22. The skin care device of claim 1, wherein the LED phototherapy appliance has a skin-contact surface area of 2 square inches to 35 square inches.

23. A method of treating the skin, comprising:
providing a skin care device comprising a skin-rejuvenation head including a first external region and a second external region, the first external region including the skin-cleansing bristles and the second external region including an LED phototherapy appliance adapted to delivery UV-light, visible light, or IR-light to a skin surface, wherein the skin-rejuvenation head includes a vibrational motor positioned therein to provide the vibratory oscillation to the skin-cleansing bristles;
scrubbing a cleanser onto a skin surface using the skin-cleansing bristles at the first external region while under vibratory oscillation provided by the vibrational motor; and
applying one or more of the UV-light, the visible light, or the IR-light using the LED phototherapy appliance to the skin surface.

24. The method of claim 23, comprising applying the visible light in the form of blue-light therapy.

25. The method of claim 24, wherein treating the skin includes treating the skin for acne.

26. The method of claim 24, wherein treating the skin includes treating the skin for bacteria.

27. The method of claim 24, wherein treating the skin includes treating the skin to calm inflammation.

28. The method of claim 24, wherein the blue-light therapy is adapted to reach beneath the skin surface.

29. The method of claim 23, comprising applying the visible light in the form of red-light therapy.

30. The method of claim 29, wherein treating the skin includes penetrating the skin surface to reach collagen and elastin beneath the skin surface.

31. The method of claim 29, wherein treating the skin includes penetrating the skin surface to rejuvenate damaged skin with broken or slowed metabolic activity.

32. The method of claim 37, wherein treating the skin includes penetrating the skin surface to rejuvenate capillaries, enhance blood flow, enhance oxygen and nutrient levels, or a combination thereof.

33. The method of claim 31, comprising applying the visible light in the form of red-light therapy and blue-light therapy simultaneously.

34. The method of claim 31, comprising applying the visible light by alternating between blue-light therapy and red-light therapy.

35. The method of claim 31, comprising applying the UV-light to the skin surface.

36. The method of claim 31, comprising applying the IR-light to the skin surface.

37. The method of claim 31, comprising selecting the intensity of UV-light, visible light, or IR-light to be applied to the skin surface.

38. The method of claim 31, wherein the vibratory oscillation provided by the vibrational motor is set at a frequency of at least 7,000 VPM.

39. The method of claim 31, wherein the vibratory oscillation provided by the vibrational motor is set at a frequency from 7,000 VPM to 12,000 VPM.

40. The method of claim 31, wherein skin care device further includes a support rod mechanically coupled to the skin-rejuvenation head.

41. The method of claim 40, further comprising a handle including a neck portion and a grip portion, wherein the support rod coupled to the handle, and wherein the neck portion is proximal to the skin-rejuvenation head and the grip portion is distal to the skin-rejuvenation head, the neck portion including an energy dampening sleeve that surrounds the support rod, leaving a gap around the support rod, and wherein the method includes dampening vibration from the vibrational motor that is translated into the handle.

42. The method of claim 41, wherein dampening the vibration is enhanced via an air gap between the energy dampening sleeve surrounding the support rod at the neck portion.
